(19) United States
(12) Patent Application Publication
(10) Pub. No.: US 2022/0267406 A1
(43) Pub. Date: Aug. 25, 2022

(54) METHOD

(71) Applicant: UCL Business Ltd., London (GB)

(72) Inventors: Hans STAUSS, London (GB); Sharyn THOMAS, London (GB); Annemarie WOOLSTON, London (GB)

(21) Appl. No.: 17/633,145

(22) PCT Filed: Aug. 6, 2020
(86) PCT No.: PCT/GB2020/051881

§ 371 Date: Feb. 4, 2022

(30) Foreign Application Priority Data
Aug. 7, 2019 (GB) 1911286.1

(51) Int. Cl. C07K 14/725; C12N 15/10

(52) U.S. Cl. CPC C07K 14/7051; C12N 15/1031; C12N 2770/40041; C12N 2799/027

(57) ABSTRACT

A method for increasing the cell surface expression and/or reducing mispairing of a TCR.

### FIELD OF THE INVENTION

The present invention relates to a T cell receptor (TCR). In particular, the present invention relates to methods for increasing cell surface expression and/or reducing mispairing of a TCR when it is expressed as an exogenous TCR, and to methods for selecting such TCR. The present invention also relates to an engineered TCR which has a high level of cell surface expression when expressed as an exogenous TCR compared to the corresponding germline TCR sequence.

### BACKGROUND TO THE INVENTION

T cell receptor (TCR) gene therapy is the transfer of antigen-specific TCR chains into recipient T-cells, thus redirecting the specificity of T-lymphocytes to target antigens of interest.

Introduced TCRs differ greatly in their ability to be expressed on the cell surface. Introduced, strongly expressed TCRs are co-expressed with the endogenous TCR or can even out-compete the endogenous TCR for cell surface expression. Introduced, weakly expressed TCRs are absent from the cell surface when co-expressed with an endogenous strong TCR (FIG. 1).

High levels of expression of an antigen specific TCR on the T cell surface results in a greater avidity of the T cell, which is beneficial for the efficacy of therapeutic TCR therapy.

WO 2016/170320 and WO 2018/073595 disclose a range of amino acid residues at certain positions which contribute to the cell surface expression of a TCR. The majority of relevant positions disclosed in each of WO 2016/170320 and WO 2018/073595 are from the TCR α chain.

There is thus a need for further methods and approaches which increase the cell surface expression of weakly expressed TCR.

### SUMMARY OF ASPECTS OF THE INVENTION

The present inventors have now determined amino acid residues present at a number of amino acid positions within the framework regions of the TCR β chain which are associated with enhanced TCR cell surface expression and/or reducing mispairing of a TCR.

The changing of an amino acid can be achieved economically, quickly and efficiently by mutagenesis, for example PCR mutagenesis. This method of increasing TCR cell surface expression does not interfere with peptide/MHC recognition by the TCR and does not affect the TCR folding or structure. Thus TCR expression is increased with a minimal impact on TCR structure.

Accordingly, the present invention provides a method for increasing the cell surface expression and/or reducing mispairing of a TCR which comprises the steps of:

(i) providing a TCR β chain sequence;

(ii) determining an amino acid residue of the TCR β chain at one or more positions selected from one or more of:
A) position 66; 67; 68; 74; 75; 76; 77; 78; 79; 80; 81; 83; 84; 85; 86; 87; 88; 89; 90; 91; 92; 93; 94; 95; 96;
B) position 1; 2; 3; 4; 5; 7; 8; 11; 12; 13; 14; 15; 16; 17; 18; 20; 22; 24; 25; 26;
C) position 39; 40; 44; 45; 46; 47; 48; 49; 50; 51; 52; 53; 54; 55;
D) position 97; 98; 99; 100; or 101; of the β chain and

(iii) altering the amino acid residue at one or more of the positions listed in step (ii) to an amino acid residue selected from:
A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;
B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;
C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;
D) arginine or serine at position 97; glutamine at position 98; threonine at position 99; serine at position 100; and methionine or valine at position 101 of the β chain.

The positions listed under group (A) are those present in framework region (FR) 3 of the β chain (based on IMGT numbering). The positions listed under group (B) are those present in FR1 of the β chain. The positions listed under group (C) are those present in FR2 of the β chain. The positions listed under group (D) are those present in FR4 of the β chain.

In one embodiment, the method is a method for increasing the cell surface expression of a T cell receptor (TCR). In one embodiment, the method is a method for increasing dimerization of TCRs, in particular dimerization of TCR α and β chains. In one embodiment, the method is a method for reducing mispairing of TCRs, in particular dimerization of introduced and endogenous TCR α and β chains.

The invention further provides a method for selecting a TCR β chain which comprises the steps of:
(i) providing one or more TCR β chain sequences; and
(ii) selecting a TCR amino acid sequence comprising one or more amino acid residues selected from one or more of:

A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;

B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;

C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;

D) arginine or serine at position 97; glutamine at position 98; threonine at position 99; serine at position 100; and methionine or valine at position 101 of the β chain.

In another aspect the present invention provides a method for selecting a TCR α chain and/or β chain which comprises the steps of:
(i) providing one or more TCR β chain sequences; and
(ii) discounting a TCR amino acid sequence comprising one or more amino acid residues selected from the residues shown in FIG. 5 or FIG. 6.

In other words, the present invention provides a method for selecting a TCR α chain and/or β chain, wherein the α chain and/or β chain is selected on the basis that it does not comprise one or more amino acid residues as shown in FIG. 5 or FIG. 6.

The invention also provides a method for determining the strength of a TCR which comprises the steps of:
(i) providing a TCR α β chain sequence; and
(ii) determining if the TCR sequence comprises one or more amino acid residues selected from one or more of:

A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;

B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;

C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;

D) arginine or serine at position 97; glutamine at position 98; threonine at position 99; serine at position 100; and methionine or valine at position 101 of the β chain.

The invention further provides an engineered TCR comprising a β chain comprising one or more of the following amino acid residues:

A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;

B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;

C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;

D) arginine or serine at position 97; glutamine at position 98; threonine at position 99; serine at position 100; and methionine or valine at position 101 of the β chain;

wherein the one or more amino acid residues is not present in the corresponding germline TCR amino acid sequence.

The invention further provides a polynucleotide encoding a TCR β chain as according to the present invention.

In another aspect the invention provides a vector comprising a polynucleotide of the invention.

In a further aspect the invention provides a cell comprising a polynucleotide or a vector according to the invention.

The invention further provides a pharmaceutical composition comprising a TCR, a polynucleotide, a vector or a cell according to the invention.

In a further aspect the present invention provides a TCR, polynucleotide, vector, cell or pharmaceutical composition according to the invention for use in treating and/or preventing a disease.

In a further aspect the present invention provides a vector or cell according to the invention for use in the manufacture of a medicament for treating and/or preventing a disease.

The invention further relates to a method for treating a disease which comprises the step of administering a TCR, polynucleotide, vector, cell or pharmaceutical composition according to the invention to a subject.

The invention further relates to a method for producing cell according to the invention, which method comprises the step of transducing a cell in vitro or ex vivo with a vector as provided by the invention.

The invention further relates to use of an engineered TCR; a polynucleotide or a vector of the invention to increase the cell surface expression of the TCR.

### DESCRIPTION OF THE FIGURES

FIG. 1—A) Depiction of a weak exogenous TCR and a strong exogenous TCR in a cell which also expresses an endogenous TCR. B) Schematic Drawing of the TCRα and TCRβ chains—SP: Signal peptide; FR: Framework Region; CDR: Complementarity Determining Region; IG: Immunoglobulin like domain of the C-region; CP: Connecting Peptide; TM: Transmembrane domain; IC: Intracellular domain.

FIG. 2—Primary T cells transduced with a modified WT1 TCR that shows TCR dominance.

FIG. 3—Amino acid residues determined to be highly significantly (p<0.00001) over-represented at recited positions in β chains of TCRs with a dominant expression profile

FIG. 4—Amino acid residues determined to be highly significantly (p<0.00001) over-represented at recited positions in a chains of TCRs with a dominant expression profile

FIG. 5—Amino acid residues determined to be highly significantly (p<0.00001) over-represented at recited positions in β chains of TCRs with a weak expression profile

FIG. 6—Amino acid residues determined to be highly significantly (p<0.00001) at recited positions in a chains of TCRs with a weak expression profile

### DETAILED DESCRIPTION

### T Cell Receptor

During antigen processing, antigens are degraded inside cells and then carried to the cell surface by major histocompatibility complex (MHC) molecules. T cells are able to recognise this peptide:MHC complex at the surface of the antigen presenting cell. There are two different classes of MHC molecules, MHC I and MHC II, that deliver peptides from different cellular compartments to the cell surface.

The T cell receptor or TCR is the molecule found on the surface of T cells that is responsible for recognizing antigens bound to MHC molecules. The TCR heterodimer consists of an α and β chain in 95% of T cells, whereas 5% of T cells have TCRs consisting of γ and δ chains.

Engagement of the TCR with antigen and MHC results in activation of its T lymphocyte through a series of biochemical events mediated by associated enzymes, co-receptors, and specialized accessory molecules.

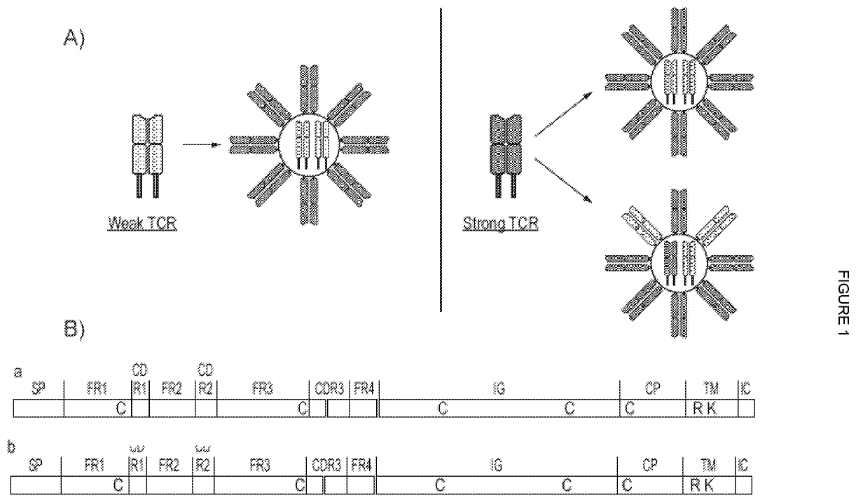

Each chain of the TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end (FIG. 1B).

The variable domain of both the TCR α-chain and β-chain have three hypervariable or complementarity determining regions (CDRs). CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. Framework regions (FRs) are positioned between the CDRs. These regions provide the structure of the TCR variable region.

The repertoire of TCR variable regions is generated by combinatorial joining of variable (V), joining (J) and diversity (D) genes; and by N region diversification (nucleotides inserted by the enzyme deoxynucleotidyl-transferase). α and γ chains are formed from recombination events between the V and J segments. β and δ chains are formed from recombination events involving the V, D and J segments.

The human TCRα locus, which also includes the TORO locus, is located on chromosome 14 (14q11.2). The TCRβ locus is located on chromosome 7 (7q34). The variable region of the TCRα chain is formed by recombination between one of 46 different Vα (variable) segments and one of 58 Jα (joining) segments (Koop et al.; 1994; Genomics; 19: 478-493). The variable region of a TCRβ chain is formed from recombination between 54 Vβ, 14 Jβ and 2 Dβ (diversity) segments (Rowen et al.; 1996; Science; 272:1755-1762).

The V and J (and D as appropriate) gene segments for each TCR chain locus have been identified and the germline sequence of each gene is known and annotated (for example see Scaviner & Lefranc; 2000; Exp Clin Immunogenet; 17:83-96 and Folch & Lefranc; 2000; Exp Clin Immunogenet; 17:42-54).

FR1, CDR1, FR2, CDR2, FR3 and CDR3 of the α chain are encoded by the Vα gene. FR4 is encoded by the Jα gene (FIG. 1B).

FR1, CDR1, FR2, CDR2 and FR3 of the β chain are encoded by the Vβ gene. CDR3 is encoded by the Dβ gene and FR4 is encoded by the Jβ gene (FIG. 1B).

As the germline sequence of each variable gene is known in the art (see Scaviner & Lefranc; as above and Folch & Lefranc; as above) the Vα and/or Vβ of a particular TCR can be sequenced and the germline V segment which is utilised in the TCR can be identified (see, for example, Hodges et al.; 2003; J Clin Pathol; 56:1-11, Zhou et al.; 2006; Laboratory Investigation; 86; 314-321).

The constant domain of the TCR domain consists of short connecting sequences in which a cysteine residue forms a disulfide bond, making a link between the two chains. The TCR of the present invention may have an additional cysteine residue in each of the α and β chains such that the TCR comprises two disulphide bonds in the constant domains (see below).

In one embodiment the constant domains employed in the TCR are human sequences.

In one embodiment the constant domains employed in the TCR are murine sequences.

The structure allows the TCR to associate with other molecules like CD3 which possess three distinct chains (γ, ϵ, and ε) in mammals and the ζ-chain. These accessory molecules have negatively charged transmembrane regions and are vital to propagating the signal from the TCR into the cell. The CD3- and ζ-chains, together with the TCR, form what is known as the T cell receptor complex.

The signal from the T cell complex is enhanced by simultaneous binding of the MHC molecules by a specific co-receptor. On helper T cells, this co-receptor is CD4 (specific for class II MHC); whereas on cytotoxic T cells, this co-receptor is CD8 (specific for class I MHC). The co-receptor not only ensures the specificity of the TCR for an antigen, but also allows prolonged engagement between the antigen presenting cell and the T cell and recruits essential molecules (e.g., LCK) inside the cell involved in the signalling of the activated T lymphocyte.

The term “T-cell receptor” is thus used in the conventional sense to mean a molecule capable of recognising a peptide when presented by an MHC molecule. The molecule may be a heterodimer of two chains α and β (or optionally γ and δ) or it may be a single chain TCR construct.

The present invention also provides the α chain or β chain from such a T cell receptor.

The present TCR may be a hybrid TCR comprising sequences derived from more than one species. For example, it has surprisingly been found that murine TCRs have been found to be more efficiently expressed in human T cells than human TCRs. The TCR may therefore comprise human variable regions and murine constant regions. A disadvantage of this approach is that the murine constant sequences may trigger an immune response, leading to rejection of the transferred T cells. However, the conditioning regimens used to prepare patients for adoptive T-cell therapy may result in sufficient immunosuppression to allow the engraftment of T cells expressing murine sequences.

The present engineered TCR comprises one or more amino acid residue as defined herein which is not encoded by the germline Vα or Vβ gene. In other words, the engineered TCR comprises an α chain and/or β chain which comprises an altered amino acid residue at one or more of the positions described herein, compared to the corresponding a chain and/or β chain as encoded by the unaltered germline Vα or Vβ gene.

The amino acid residues identified herein are numbered according to the International ImMunoGeneTics information system′ (IMGT). This system is well known in the art (Lefrance et al.; 2003; Dev Comp Immunol; 27: 55-77) and is based on the high conservation of the structure of the variable region. The numbering takes into account and combines the definition of the FR and CDRs, structural data from X-ray diffraction studies and the characterization of the hypervariable loops.

The delimitations of the FR and CDR regions are defined within the IGMT numbering system. The FR1 region comprises positions 1-26 (25-26 amino acids, depending on the V-GENE group or subgroup) with 1st-CYS at position 23. The FR2 region comprises positions 39-55 (16-17 amino acids) with a conserved TRP at position 41. The FR3 region comprises positions 66-104 (36-39 amino acids, depending on the VGENE group or subgroup) with a conserved hydrophobic amino acid at position 89 and the 2nd-CYS at position 104. Residue 1 of the IGMT numbering system is the first residue in FR1. Residue 104 of the IGMT numbering system is the last residue in FR3.

As such, the numbering system used herein refers to the position of the amino acid within the entire α chain or the entire β chain, as appropriate.

The IMGT numbering therefore allows a standardized description of amino acid positions within TCR variable regions and comparisons with the germline encoded sequences to be performed.

Methods suitable for generating an engineered TCR according to the present invention are known in the art.

For example mutagenesis may be performed to alter specific nucleotides in a nucleic acid sequence encoding the TCR. Such mutagenesis will alter the amino acid sequence of the TCR so that it comprises one or more of the amino acid residues as described herein.

An example of a mutagenesis method is the Quikchange method (Papworth et al.; 1996; Strategies; 9(3); 3-4). This method involves the use of a pair of complementary mutagenic primers to amplify a template nucleic acid sequence in a thermocycling reaction using a high-fidelity non-strand-displacing DNA polymerase, such as pfu polymerase.

The present inventors have determined that the presence of particular amino acid residues in specific positions of the TCR framework regions is associated with the level of cell surface expression of the TCR.

As used herein, cell surface expression is synonymous with expression strength. As such, ‘strong’ or ‘high’ expression is equivalent to high levels of cell surface expression of the TCR. ‘Weak’ or ‘low’ expression is equivalent to low levels of cell surface expression of the TCR.

Increasing the cell surface expression of a TCR means that a TCR comprising one or more amino acid residues as described herein has a higher level of cell surface expression relative to an equivalent TCR comprising the amino acid sequence encoded by the germline sequence. An equivalent TCR comprising the amino acid sequence encoded by the germline sequence refers to a TCR which has not been altered to comprise a non-germline amino acid residue at a given position as described herein—i.e. the unaltered TCR has the wild-type amino acid residue at the specific position.

In one embodiment the present engineered TCR may have a cell surface expression which is at least 1.5-, 2-, 2.5-, 3-, 4-, 5-, 6-, 7-, 8-, 9-, or 10-fold greater than the corresponding TCR comprising the unmodified germline TCR sequence.

Cell surface expression of a TCR may be determined by methods which are known in the art. For example, the cell surface expression of a TCR may be determined using conventional flow cytometry methods known in the art (see, for example, Shapiro; Practical Flow Cytometry; John 2005; Science).

For example, the cell surface expression of a TCR may be expressed as the mean fluorescent intensity (MFI) of TCR expression in a population of cells (see Shapiro; as above). In one embodiment the MFI of a population of cells expressing the present engineered TCR may be at least 1.5-, 2-, 2.5-, 3-, 4-, 5-, 6-, 7-, 8-, 9-, or 10-fold greater than the MFI of an corresponding population of cells expressing the corresponding TCR comprising the unmodified germline TCR sequence.

The cell surface expression of a TCR may be expressed as the percentage of cells within a population which express the TCR at the cell surface. Such a percentage may be determined using conventional flow cytometry methods as is well known in the art (see, for example, Shapiro; as above and Henel et al.; 2007; Lab Medicine; 38; 7; 428-436).

In one embodiment, the present engineered TCR may be expressed by at least 2, at least 5, at least 10, at least 15, at least 20, at least 25, at least 30, at least 40, at least 50, at least 60 or at least 70% more cells in a population compared to the corresponding TCR comprising the unmodified germline TCR sequence.

In one embodiment, the cell surface expression of the engineered TCR is increased compared to the corresponding TCR comprising the unmodified germline TCR sequence but the relative level of the mRNA encoding the engineered TCR or the unmodified germline TCR are essentially the same. As used herein, “essentially the same” may mean that mRNA levels differ by, for example, less than 1.5 fold. Relative mRNA levels may be determined using methods which are known in the art; for example RT-qPCR, northern blotting and flow cytometry RNA assays.

The methods and TCRs of the present invention may enhance dimerization of the TCR α chain and β chain. In particular, the methods and TCRs of the present invention may enhance the dimerization of the present a chain and β chain when they are expressed recombinantly, e.g., as an exogenous TCR in a cell (e.g. a cell as described herein).

The methods and TCRs of the present invention may reduce mispairing between the TCR α chain and/or β chain of the present invention. In particular, the methods and TCRs of the present invention may reduce mispairing between the present TCR α chain and/or β chain and an endogenous α chain and/or β chain of a cell.

The methods and TCRs of the present invention may enhance the dimerization of the TCR α chain and β chain and reduce mispairing between the TCR α chain and/or β chain of the present invention.

Without wishing to be bound by theory, the present inventors consider that the enhanced dimerization and/or reduced mispairing described herein may e.g. contribute to the increased cell surface expression provided by the present invention. Such effects are similar in character to those described for other technologies, such as the introduction of an engineered second disulphide bond into the constant region of an exogenous TCR. Accordingly, the methods and TCRs of the present invention provide further methods for enhancing the dimerization and/or reducing the mispairing of a TCR α chain and β chain when they are expressed recombinantly, e.g., as an exogenous TCR in a cell.

Suitably the present engineered TCR comprises one or more of following altered amino acid residues selected from:

A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;

B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;

C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;

D) arginine or serine at position 97; glutamine at position 98; threonine at position 99; serine at position 100; and methionine or valine at position 101 of the β chain;

wherein the one or more amino acid residues is not present in the corresponding germline TCR amino acid sequence.

The one or more altered amino acid residues may be selected from Group (A) consisting of lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain.

The one or more altered amino acid residues may be selected from Group (B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain.

The one or more altered amino acid residues may be selected from Group (C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain.

The one or more altered amino acid residues may be selected from Group (D) arginine or serine at position 97; glutamine at position 98; threonine at position 99; serine at position 100; and methionine or valine at position 101 of the β chain.

The altered amino acid introduced may be lysine or leucine at position 66 of the β chain. The altered amino acid introduced may be lysine at position 66 of the β chain. The altered amino acid introduced may leucine at position 66 of the β chain.

The altered amino acid introduced may be glutamic acid at position 67 of the β chain.

The altered amino acid introduced may be lysine at position 68 of the β chain.

The altered amino acid introduced may be glutamic acid, asparagine or glycine at position 74 of the β chain. The altered amino acid introduced may be glutamic acid at position 74 of the β chain. The altered amino acid introduced may be asparagine at position 74 of the β chain. The altered amino acid introduced may be glycine at position 74 of the β chain.

The altered amino acid introduced may be glycine at position 75 of the β chain.

The altered amino acid introduced may be tyrosine at position 76 of the β chain.

The altered amino acid introduced may be asparagine, valine or lysine at position 77 of the β chain. The altered amino acid introduced may be asparagine at position 77 of the β chain. The altered amino acid introduced may be valine at position 77 of the β chain. The altered amino acid introduced may lysine at position 77 of the β chain.

The altered amino acid introduced may be valine or glycine at position 78 of the β chain. The altered amino acid introduced may be valine at position 78 of the β chain. The altered amino acid introduced may be glycine at position 78 of the β chain.

The altered amino acid introduced may be serine or arginine at position 79 of the β chain. The altered amino acid introduced may be serine at position 79 of the β chain. The altered amino acid introduced may be arginine at position 79 of the β chain.

The altered amino acid introduced may be arginine or serine at position 80 of the β chain. The altered amino acid introduced may be arginine at position 80 of the β chain. The altered amino acid introduced may be serine at position 80 of the β chain.

The altered amino acid introduced may be glutamic acid, leucine, serine or lysine at position 81 of the β chain. The altered amino acid introduced may be glutamic acid at position 81 of the β chain. The altered amino acid introduced may be leucine at position 81 of the β chain. The altered amino acid introduced may be serine at position 81 of the β chain. The altered amino acid introduced may be lysine at position 81 of the β chain.

The altered amino acid introduced may be lysine or serine at position 83 of the β chain. The altered amino acid introduced may be lysine at position 83 of the β chain. The altered amino acid introduced may be serine at position 83 of the β chain.

The altered amino acid introduced may be lysine or asparagine at position 84 of the β chain. The altered amino acid introduced may be lysine at position 84 of the β chain. The altered amino acid introduced may be asparagine at position 84 of the β chain.

The altered amino acid introduced may be glutamic acid or arginine at position 85 of the β chain. The altered amino acid introduced may be glutamic acid at position 85 of the β chain. The altered amino acid introduced may be arginine at position 85 of the β chain.

The altered amino acid introduced may be arginine or glutamic acid at position 86 of the β chain. The altered amino acid introduced may be arginine at position 86 of the β chain. The altered amino acid introduced may be glutamic acid at position 86 of the β chain.

The altered amino acid introduced may be phenylalanine at position 87 of the β chain.

The altered amino acid introduced may be serine, leucine or asparagine at position 88 of the β chain. The altered amino acid introduced may be serine at position 88 of the β chain. The altered amino acid introduced may be leucine at position 88 of the β chain. The altered amino acid introduced may be asparagine at position 88 of the β chain.

The altered amino acid introduced may be methionine at position 89 of the β chain.

The altered amino acid introduced may be isoleucine, arginine or glutamic acid at position 90 of the β chain. The altered amino acid introduced may be isoleucine acid at position 90 of the β chain. The altered amino acid introduced may be arginine at position 90 of the β chain. The altered amino acid introduced may be glutamic acid at position 90 of the β chain.

The altered amino acid introduced may be leucine at position 91 of the β chain.

The altered amino acid introduced may be glutamic acid or asparagine at position 92 of the β chain. The altered amino acid introduced may be glutamic acid at position 92 of the β chain. The altered amino acid introduced may be asparagine at position 92 of the β chain.

The altered amino acid introduced may be serine or leucine at position 93 of the β chain. The altered amino acid introduced may be serine at position 93 of the β chain. The altered amino acid introduced may be leucine at position 93 of the β chain.

The altered amino acid introduced may be alanine at position 94 of the β chain.

The altered amino acid introduced may be alanine, serine or threonine at position 95 of the β chain. The altered amino acid introduced may be alanine at position 95 of the β chain. The altered amino acid introduced may be serine at position 95 of the β chain. The altered amino acid introduced may be threonine at position 95 of the β chain.

The altered amino acid introduced may be threonine or serine at position 96 of the β chain. The altered amino acid introduced may be threonine at position 96 of the β chain. The altered amino acid introduced may be serine at position 96 of the β chain.

The altered amino acid introduced may be aspartic acid, asparagine or lysine at position 1 of the β chain. The altered amino acid introduced may be aspartic acid at position 1 of the β chain. The altered amino acid introduced may be asparagine at position 1 of the β chain. The altered amino acid introduced may be lysine at position 1 of the β chain.

The altered amino acid introduced may be valine at position 2 of the β chain.

The altered amino acid introduced may be lysine, alanine or glutamine at position 3 of the β chain. The altered amino acid introduced may be lysine at position 3 of the β chain. The altered amino acid introduced may be alanine at position 3 of the β chain. The altered amino acid introduced may be glutamine at position 3 of the β chain.

The altered amino acid introduced may be valine at position 4 of the β chain.

The altered amino acid introduced may be threonine at position 5 of the β chain.

The altered amino acid introduced may be serine or asparagine at position 7 of the β chain. The altered amino acid introduced may be serine at position 7 of the β chain. The altered amino acid introduced may be asparagine at position 7 of the β chain.

The altered amino acid introduced may be serine at position 8 of the β chain.

The altered amino acid introduced may be leucine at position 11 of the β chain.

The altered amino acid introduced may be valine at position 12 of the β chain.

The altered amino acid introduced may be lysine, leucine or alanine at position 13 of the β chain. The altered amino acid introduced may be lysine at position 13 of the β chain. The altered amino acid introduced may be leucine at position 13 of the β chain. The altered amino acid introduced may be alanine at position 13 of the β chain.

The altered amino acid introduced may be arginine, lysine, threonine or valine at position 14 of the β chain. The altered amino acid introduced may be arginine at position 14 of the β chain. The altered amino acid introduced may be lysine at position 14 of the β chain. The altered amino acid introduced may be threonine at position 14 of the β chain. The altered amino acid introduced may be valine at position 14 of the β chain.

The altered amino acid introduced may be threonine or isoleucine at position 15 of the β chain. The altered amino acid introduced may be threonine at position 15 of the β chain. The altered amino acid introduced may be isoleucine at position 15 of the β chain.

The altered amino acid introduced may be glycine or serine at position 16 of the β chain. The altered amino acid introduced may be glycine at position 16 of the β chain. The altered amino acid introduced may be serine at position 16 of the β chain.

The altered amino acid introduced may be glutamic acid at position 17 of the β chain.

The altered amino acid introduced may be lysine, glutamine, asparagine or alanine at position 18 of the β chain. The altered amino acid introduced may be lysine at position 18 of the β chain. The altered amino acid introduced may be glutamine at position 18 of the β chain. The altered amino acid introduced may be asparagine at position 18 of the β chain. The altered amino acid introduced may be alanine at position 18 of the β chain.

The altered amino acid introduced may be phenylalanine at position 20 of the β chain.

The altered amino acid introduced may be glutamic acid, methionine or threonine at position 22 of the β chain. The altered amino acid introduced may be glutamic acid at position 22 of the β chain. The altered amino acid introduced may be methionine at position 22 of the β chain. The altered amino acid introduced may be threonine at position 22 of the β chain.

The altered amino acid introduced may be valine, alanine or histidine at position 24 of the β chain. The altered amino acid introduced may be valine at position 24 of the β chain. The altered amino acid introduced may be alanine at position 24 of the β chain. The altered amino acid introduced may be histidine at position 24 of the β chain.

The altered amino acid introduced may be glutamine at position 25 of the β chain.

The altered amino acid introduced may be aspartic acid or asparagine at position 26 of the β chain. The altered amino acid introduced may be aspartic acid at position 26 of the β chain. The altered amino acid introduced may be asparagine at position 26 of the β chain.

The altered amino acid introduced may be methionine at position 39 of the β chain.

The altered amino acid introduced may be phenylalanine or serine at position 40 of the β chain. The altered amino acid introduced may be phenylalanine at position 40 of the β chain. The altered amino acid introduced may be serine at position 40 of the β chain.

The altered amino acid introduced may be glutamine at position 44 of the β chain.

The altered amino acid introduced may be aspartic acid or threonine at position 45 of the β chain. The altered amino acid introduced may be aspartic acid at position 45 of the β chain. The altered amino acid introduced may be threonine at position 45 of the β chain.

The altered amino acid introduced may be proline at position 46 of the β chain.

The altered amino acid introduced may be glycine at position 47 of the β chain.

The altered amino acid introduced may be leucine, methionine or histidine at position 48 of the β chain. The altered amino acid introduced may be leucine at position 48 of the β chain. The altered amino acid introduced may be methionine at position 48 of the β chain. The altered amino acid introduced may be histidine at position 48 of the β chain.

The altered amino acid introduced may be glycine or phenylalanine at position 49 of the β chain. The altered amino acid introduced may be glycine at position 49 of the β chain. The altered amino acid introduced may be phenylalanine at position 49 of the β chain.

The altered amino acid introduced may be leucine at position 50 of the β chain.

The altered amino acid introduced may be arginine at position 51 of the β chain.

The altered amino acid introduced may be leucine, isoleucine or glutamine at position 52 of the β chain. The altered amino acid introduced may be leucine at position 52 of the β chain. The altered amino acid introduced may be isoleucine at position 52 of the β chain. The altered amino acid introduced may be glutamine at position 52 of the β chain.

The altered amino introduced acid may be isoleucine at position 53 of the β chain.

The altered amino acid introduced may be tyrosine or threonine at position 54 of the β chain. The altered amino acid introduced may be tyrosine at position 54 of the β chain. The altered amino acid introduced may be threonine at position 54 of the β chain.

The altered amino acid introduced may be phenylalanine or glutamic acid at position 55 of the β chain. The altered amino acid introduced may be phenylalanine at position 55 of the β chain. The altered amino acid introduced may be glutamic acid at position 55 of the β chain.

The altered amino acid introduced may be arginine or serine at position 97 of the β chain. The altered amino acid introduced may be arginine at position 97 of the β chain. The altered amino acid introduced may be serine at position 97 of the β chain.

The altered amino acid introduced may be glutamine at position 98 of the β chain.

The altered amino acid introduced may be threonine at position 99 of the β chain.

The altered amino acid introduced may be serine at position 100 of the β chain.

The altered amino acid introduced may be methionine or valine at position 101 of the β chain. The altered amino acid introduced may be methionine at position 101 of the β chain. The altered amino acid introduced may be valine at position 101 of the β chain.

The term “one or more” as used herein may include one, two, three, four, five, six, seven, eight, nine, ten, eleven, twelve, thirteen, fourteen, fifteen, sixteen, seventeen, eighteen, nineteen, twenty or more amino acid residues as described herein.

Each of the one or more altered amino acid residues may be selected from any one of Groups (A), (B), (C) and (D) as defined herein.

Each of the one or more altered amino acid residues may be selected from Group (A) as defined herein.

Each of the one or more altered amino acid residues may be selected from Group (B) as defined herein.

Each of the one or more altered amino acid residues may be selected from Group (C) as defined herein.

Each of the one or more altered amino acid residues may be selected from Group (D) as defined herein.

The term “two or more” as used herein may include two, three, four, five, six, seven, eight, nine, ten, eleven, twelve, thirteen, fourteen, fifteen, sixteen, seventeen, eighteen, nineteen, twenty or more amino acid residues as described herein.

The engineered TCR may comprise a plurality of the amino acid residues recited above. In other words, the TCR may comprise at least 2, at least 3, at least 4, at least 5, at least 6, at least 7, at least 8, at least 9, at least 10, at least 15 or at least 20 of the amino acid residues recited above.

### Nucleic Acid Sequence

The present invention further provides a nucleotide sequence encoding an engineered TCR receptor as described herein or a part thereof, for example the variable sequence of the α chain or the β chain; the α chain and/or the β chain.

As used herein, the terms “polynucleotide” and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The polynucleotide may be double or single stranded, and may be RNA or DNA.

The polynucleotide may be codon optimised. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation may also involve the removal of mRNA instability motifs and cryptic splice sites.

The polynucleotide may comprise a nucleic acid sequence which enables both a nucleic acid sequence encoding an α chain and a nucleic acid sequence a β chain to be expressed from the same mRNA transcript.

For example, the polynucleotide may comprise an internal ribosome entry site (IRES) between the nucleic acid sequences which encode the α chain and the β chain. An IRES is a nucleotide sequence that allows for translation initiation in the middle of a mRNA sequence.

The polynucleotide may comprise a nucleic acid sequence encoding an α chain and a nucleic acid sequence a β chain linked by an internal self-cleaving sequence.

The internal self-cleaving sequence may be any sequence which enables the polypeptide comprising the α chain and the polypeptide comprising the β chain to become separated.

The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The self-cleaving peptide may be a 2A self-cleaving peptide from an aphtho- or a cardiovirus.

### Vector

The present invention also provides a vector comprising a nucleotide sequence as described herein.

The term “vector” includes an expression vector, i.e. a construct capable of in vivo or in vitro/ex vivo expression. Also encompassed are cloning vectors.

Viral delivery systems include but are not limited to adenovirus vector, an adeno-associated viral (AAV) vector, a herpes viral vector, retroviral vector, lentiviral vector, baculoviral vector.

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al (“Retroviruses” 1997 Cold Spring Harbour Laboratory Press Eds: J M Coffin, S M Hughes, H E Varmus pp 758-763).

Lentiviruses also belong to the retrovirus family, but they can infect both dividing and non-dividing cells (Lewis et al (1992) EMBO J. 3053-3058).

The vector may be capable of transferring a nucleotide according to the second aspect of the invention to a cell, such as a T-cell. The vector should ideally be capable of sustained high-level expression in T cells, so that the introduced TCR may compete successfully with the endogenous TCR for a limited pool of CD3 molecules.

The vector may be a retroviral vector. The vector may be based on or derivable from the MP71 vector backbone. The vector may lack a full-length or truncated version of the Woodchuck Hepatitis Response Element (WPRE).

For efficient infection of human cells, viral particles may be packaged with amphotropic envelopes or gibbon ape leukemia virus envelopes.

Increasing the supply of CD3 molecules may increase TCR expression in gene modified cells. The vector may therefore also comprise the genes for CD3-gamma, CD3-delta, CD3-epsilon and/or CD3-zeta. The vector may just comprise the gene for CD3-zeta. The genes may be linked by self-cleaving sequences, such as the 2A self-cleaving sequence. Alternatively one or more separate vectors may be provided encoding CD3 gene for co-transfer with the TCR-encoding vector(s).

cell

The present invention further relates to a cell which comprises a polynucleotide according to the present invention. The cell may express a T-cell receptor of the invention.

The cell may a mammalian cell, in particular a human cell.

The cell may be a T-cell. The T-cell may be any T-cell subset, including for example, αβ T-cell, γδ T-cell or regulatory T cells.

Suitably, the T cell may be a regulatory T cell.

The cell may be a natural killer cell.

The cell may be a natural killer T cell.

The cell may be a progenitor cell or a stem cell that can develop into a T cell

The cell may be an induced pluripotent stem cells that can develop into a T cell

The cell may be derived from a cell isolated from a subject. The cell may be part of a mixed cell population isolated from the subject, such as a population of peripheral blood lymphocytes (PBL).

T cells within the PBL population may be activated by methods known in the art, such as using anti-CD3 and CD28 antibodies.

The T-cell may be a CD4+ helper T cell or a CD8+ cytotoxic T cell. The cell may be in a mixed population of CD4+ helper T cell/CD8+ cytotoxic T cells. Polyclonal activation, for example using anti-CD3 antibodies optionally in combination with anti-CD28 antibodies will trigger the proliferation of CD4+ and CD8+ T cells, but may also trigger the proliferation of CD4+25+ regulatory T-cells.

In one embodiment the T cell is a CD8+ cytotoxic T cell.

In one embodiment a cell which expresses an engineered TCR according to the present invention may have increased functional activity compared to a cell which expresses the corresponding TCR comprising the unmodified germline TCR sequence.

Increased functional activity may refer, for example, to increased cytokine production by the cell following binding of antigen to the TCR. The cytokine may be selected from IFNγ, IL-2, GM-CSF, TNFalpha, IL-4, IL-5, IL-6, IL-9, IL-10 and IL-13.

The cytokine may be IFNγ and/or IL-2.

As used herein “increased cytokine production” means that—upon binding of antigen to the TCR—the amount of cytokine produced by a cell which expresses an engineered TCR according to the present invention in greater than the amount produced by an equivalent cell which expresses the corresponding TCR comprising the unmodified germline TCR sequence.

The amount of cytokine produced by a cell which expresses an engineered TCR according to the present invention may be at least 1.5-, 2-, 2.5-, 3-, 4-, 5-, 10-, 20- or 50-fold greater than that produced by an equivalent cell which expresses the corresponding TCR comprising the unmodified germline TCR sequence.

The amount of cytokine produced by a cell may be determined using methods which are known in the art—for example flow cytometry or ELISA.

The present invention also provides a method of producing a cell according to the invention which comprises the step of transfecting or transducing a cell in vitro or ex vivo with a vector according to the invention.

The cell may be isolated from the subject to which the genetically modified cell is to be adoptively transferred. In this respect, the cell may be made by isolating a T-cell from a subject, optionally activating the T-cell, TCR gene transfer ex vivo and subsequent immunotherapy of the subject by adoptive transfer of the TCR-transduced cells.

Alternatively, the cell may be isolated from a different subject, such that it is allogeneic. The cell may be isolated from a donor subject. For example, if the subject is undergoing allogeneic haematopoietic stem cell transplantation (Allo-HSCT), the cell may be derived from the donor, from which the HSCs are derived. If the subject is undergoing or has undergone solid organ transplant, the cell may be derived from the subject from whom the solid organ was derived.

Alternatively, the cell may be, or be derived from, a stem cell, such as a haemopoietic stem cell (HSC). Gene transfer into HSCs does not lead to TCR expression at the cell surface as stem cells do not express the CD3 molecules. However, when stem cells differentiate into lymphoid precursors that migrate to the thymus, the initiation of CD3 expression leads to the surface expression of the introduced TCR in thymocytes.

An advantage of this approach is that the mature T cells, once produced, express only the introduced TCR and little or no endogenous TCR chains, because the expression of the introduced TCR chains suppresses rearrangement of endogenous TCR gene segments to form functional TCR alpha and beta genes.

A further benefit is that the gene-modified stem cells are a continuous source of mature T-cells with the desired antigen specificity. The cell may therefore be a gene-modified stem cell, which, upon differentiation, produces a T-cell expressing a TCR of the first aspect of the invention. The present invention also provides a method of producing a T-cell expressing a TCR of the invention by inducing the differentiation of a stem cell which comprises a polynucleotide of the invention.

A disadvantage of the stem cell approach is that TCRs with the desired specificity may get deleted during T-cell development in the thymus or may induce tolerance when expressed in peripheral T-cells. Another possible issue is the risk of insertional mutagenesis in stem cells.

### Pharmaceutical Composition

The present invention further provides a pharmaceutical composition comprising a TCR, polynucleotide, vector or a cell according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Use

The present invention further relates to TCR, polynucleotide, vector, cell or pharmaceutical composition of the present invention for use in treating and/or preventing a disease. In particular, the present invention provides a pharmaceutical composition comprising a cell according to the invention for use in treating and/or preventing a disease.

‘Treating’ relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

‘Preventing’ relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The present invention also provides the use of an engineered TCR; a nucleic acid sequence or a vector according to the present invention to increase the cell surface expression of the TCR.

The present invention also provides the use of an engineered TCR; a nucleic acid sequence or a vector according to the present invention to increase the functional activity of a cell. The functional activity may be any functional activity as described herein.

### Method of Treatment

The present invention provides a method for treating and/or preventing a disease which comprises the step of administering the cells of the present invention (for example in a pharmaceutical composition as described above) to a subject.

The method may involve the steps of:
(i) isolated a cell-containing sample;
(ii) transducing or transfecting such cells with a nucleic acid sequence or vector provided by the present invention;
(iii) administering the cells from (ii) to a subject.

The present invention also provides a cell or a TCR of the present invention for use in treating and/or preventing a disease.

In a preferred embodiment of the present invention, the subject of any of the methods described herein is a mammal, preferably a cat, dog, horse, donkey, sheep, pig, goat, cow, mouse, rat, rabbit or guinea pig, but most preferably the subject is a human.

For administration of cells as described herein, any mode of administration of the cell population which is common or standard in the art may be used, e.g. injection or infusion, by an appropriate route. In one aspect up to 5×10⁸ cells are administered per kg in humans. Thus, for example, a human with a body weight of 100 kg may receive a dose of up to 5×10¹⁰ cells per treatment. The dose can be repeated at later times if necessary.

The invention also relates to the use of a TCR, a vector or a cell according to the present invention in the manufacture of a medicament for treating and/or preventing a disease.

### Disease

The disease to be treated and/or prevented by the methods and uses of the present invention may be any disease which induces a T cell mediated immune response.

For example, the disease to be treated and/or prevented by the methods and uses of the present invention may be cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease to be treated and/or prevented by the methods and uses of the present invention may be an infection, for example a bacterial or viral infection.

The disease to be treated and/or prevented by the methods and uses of the present invention may be an autoimmune disease such as type 1 diabetes, multiple sclerosis, inflammatory bowel disease, rheumatoid arthritis.

The present invention further provides a method for inducing tolerance to a transplant; treating and/or preventing cellular and/or humoral transplant rejection; treating and/or preventing graft-versus-host disease (GvHD).

### Method

The present invention provides a method for increasing the cell surface expression and/or reducing mispairing of a TCR which comprises the steps of:
(i) providing a TCR β chain sequence;
(ii) determining an amino acid residue of the TCR β chain at one or more positions selected from one or more of:

A) position 66; 67; 68; 74; 75; 76; 77; 78; 79; 80; 81; 83; 84; 85; 86; 87; 88; 89; 90; 91; 92; 93; 94; 95; 96;

B) position 1; 2; 3; 4; 5; 7; 8; 11; 12; 13; 14; 15; 16; 17; 18; 20; 22; 24; 25; 26;

C) position 39; 40; 44; 45; 46; 47; 48; 49; 50; 51; 52; 53; 54; 55;

D) position 97; 98; 99; 100; or 101; of the β chain; and
(iii) altering the amino acid residue at one or more of the positions listed in step (ii) to an amino acid residue selected from:

A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;

B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;

C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;

D) arginine or serine at position 97; glutamine at position 98; threonine at position 99; serine at position 100; and methionine or valine at position 101 of the β chain.

Accordingly, the altered TCR generated in by altering one or more amino acids as defined in step (iii) is an engineered TCR as referred to herein.

As described herein, the germline sequence of each variable gene is known in the art (see Scaviner & Lefranc; as above and Folch & Lefranc; as above) and the Vα and/or Vβ germline V segment utilised in a TCR can therefore by determined by sequencing and comparing to the known germline sequences (see, for example, Hodges et al.; as above, Zhou et al.; 2006; as above). The present step of determining an amino acid residue of the TCR α chain and/or β chain at one or more positions therefore specifically relates to determining the amino acid residue that is present at one or more of the particular positions described herein.

The method may comprise determining the amino acid residue present at any position or plurality of positions as described herein.

The amino acid sequence of the TCR may be altered such that it comprises any amino acid residue or any plurality of amino acid residues as described herein.

The amino acid sequence of the TCR may be altered such that it comprises one or more amino acid residues as defined above.

The amino acid sequence of a TCR may be altered using methods which are well known in the art. For example, the amino acid sequence of the TCR may be altered mutagenesis of a nucleic acid sequence encoding the TCR.

As detailed above, increasing the cell surface expression of a TCR means that a TCR comprising at least one amino acid residue according to the present invention has a higher level of cell surface expression relative to an equivalent TCR comprising the amino acid sequence encoded by the germline sequence. An equivalent TCR comprising the amino acid sequence encoded by the germline sequence refers to a TCR which has not been altered to comprise an amino acid residue according to the present invention—i.e. the unaltered TCR has the wild-type amino acid residue at the specific position.

Cell surface expression of a TCR may be determined using any of the methods described herein.

The present invention further relates to a method for selecting a TCR which, when expressed in a cell, has a high level of cell surface expression; which method comprises the steps of:
(i) providing one or more TCR β chain sequences; and
(ii) selecting a TCR amino acid sequence comprising one or more amino acid residues selected from one or more of:

A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;

B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;

C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;

D) arginine or serine at position 97; glutamine at position 98; threonine at position 99; serine at position 100; and methionine or valine at position 101 of the β chain.

The method may comprise the step of selecting a TCR which comprises one or more amino acid residues as defined herein.

In a further aspect the present invention provides a method for determining the strength of a TCR which comprises the steps of:
(i) providing a TCR α chain and/or β chain sequence; and
(ii) determining if the TCR sequence comprises one or more amino acid residues selected from one or more of:

A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;

B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;

C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;

D) arginine or serine at position 97; glutamine at position 98; threonine at position 99; serine at position 100; and methionine or valine at position 101 of the β chain.

The α chain and/or β chain sequence may be an amino acid sequence or a polynucleotide sequence which encodes an a chain and/or a β chain.

The method may comprise may comprise determining the amino acid residue present at one or more positions as described herein.

Such a method is useful for predicting the level of surface expression of a TCR, for example a therapeutic TCR, when it is expressed as an exogenous TCR in a cell, for example a T cell.

The present invention also relates to a method for increasing dimerization of TCR α and β chains which comprises the steps of:
(i) providing a TCR β chain sequence;
(ii) determining an amino acid residue of the TCR β chain at one or more positions selected from one or more of:

A) position 66; 67; 68; 74; 75; 76; 77; 78; 79; 80; 81; 83; 84; 85; 86; 87; 88; 89; 90; 91; 92; 93; 94; 95; 96;

B) position 1; 2; 3; 4; 5; 7; 8; 11; 12; 13; 14; 15; 16; 17; 18; 20; 22; 24; 25; 26;

C) position 39; 40; 44; 45; 46; 47; 48; 49; 50; 51; 52; 53; 54; 55;

D) position 97; 98; 99; 100; or 101; of the β chain; and
(iii) altering the amino acid residue at one or more of the positions listed in step (ii) to an amino acid residue selected from: A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;

B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;

C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;

D) arginine or serine at position 97; glutamine at position 98; threonine at position 99; serine at position 100; and methionine or valine at position 101 of the β chain.

In some embodiments, the TCR α and β chains to be altered by said method for increasing dimerization of TCR α and β chains are of a soluble TCR (sTCR). sTCR typically comprise the TCR variable domains and at least part of the constant domain but lack the transmembrane domain and intracellular, cytoplasmic domain. Suitably, the sTCR of the present method comprises (i) all or part of a TCR α chain, except the transmembrane domain thereof, and (ii) all or part of a TCR β chain, except the transmembrane domain therefore, wherein (i) and (ii) each comprise a functional variable domain and at least a part of the constant domain of the TCR. In one aspect, a sTCR of the present method does not comprise a transmembrane domain. Thus, the sTCR is not anchored on the surface of cell. Suitably, the sTCR α chain and β chain may comprise all of the extracellular constant Ig domain of the TCR chain. Suitably, the sTCR α chain and β chain may comprise all of the extracellular domain of the TCR chain.

Also provided is a TCR or a sTCR according to the method for increasing dimerization of TCR α and β chains described above.

Such method may improve expression yield of the TCR on the cell surface or of the sTCR being excreted as soluble protein by the cell.

### Further Aspects of the Invention

Further aspects of the present invention are defined in the following numbered paragraphs:

Paragraph 1. A method for increasing the cell surface expression and/or reducing mispairing of a TCR which comprises the steps of:
(i) providing a TCR α chain sequence;
(ii) determining an amino acid residue of the TCR α chain at one or more positions selected from 1, 2, 4, 10, 12, 13, 14, 17, 25, 43, 44, 45, 46, 54, 77, 78, 80, 82, 84, 85, 87, 88, 95, 97 and 99:

and
(iii) altering the amino acid residue at one or more of the positions listed in step (ii) to an amino acid residue selected from:

glutamine or glycine at position 1,

isoleucine or lysine at position 2,

valine at position 4,

aspartic acid, proline, glutamine or glutamic acid at position 10,

serine at position 12,

leucine or valine at position 13,

alanine or proline at position 14,

asparagine at position 17,

tyrosine at position 25,

histidine at position 43,

glutamine at position 44,

tyrosine, asparagine or proline at position 45,

leucine or serine at position 46,

methionine or phenylalanine at position 54,

alanine or serine at position 77,

alanine at position 78,

threonine or valine at position 80,

lysine at position 82,

serine, alanine or threonine at position 84,

arginine or threonine at position 85,

valine or phenylalanine at position 87,

histidine or leucine at position 88,

leucine or glutamine at position 95,

glutamic acid or threonine at position 97, and

serine at position 99.

Paragraph 2. A method for selecting a TCR which comprises the steps of:
(i) providing one or more TCR α chain sequences; and
(ii) selecting a TCR amino acid sequence comprising one or more amino acid residues selected from: glutamine or glycine at position 1, isoleucine or lysine at position 2, valine at position 4, aspartic acid, proline, glutamine or glutamic acid at position 10, serine at position 12, leucine or valine at position 13, alanine or proline at position 14, asparagine at position 17, tyrosine at position 25, histidine at position 43, glutamine at position 44, tyrosine, asparagine or proline at position 45, leucine or serine at position 46, methionine or phenylalanine at position 54, alanine or serine at position 77, alanine at position 78, threonine or valine at position 80, lysine at position 82, serine, alanine or threonine at position 84, arginine or threonine at position 85, valine or phenylalanine at position 87, histidine or leucine at position 88, leucine or glutamine at position 95, glutamic acid or threonine at position 97, and serine at position 99.

Paragraph 3. A method for determining the strength of a TCR which comprises the steps of:
(i) providing a TCR α chain sequence; and
(ii) determining if the TCR sequence comprises one or more amino acid residues selected from: glutamine or glycine at position 1, isoleucine or lysine at position 2, valine at position 4, aspartic acid, proline, glutamine or glutamic acid at position 10, serine at position 12, leucine or valine at position 13, alanine or proline at position 14, asparagine at position 17, tyrosine at position 25, histidine at position 43, glutamine at position 44, tyrosine, asparagine or proline at position 45, leucine or serine at position 46, methionine or phenylalanine at position 54, alanine or serine at position 77, alanine at position 78, threonine or valine at position 80, lysine at position 82, serine, alanine or threonine at position 84, arginine or threonine at position 85, valine or phenylalanine at position 87, histidine or leucine at position 88, leucine or glutamine at position 95, glutamic acid or threonine at position 97, and serine at position 99.

Paragraph 4. An engineered T cell receptor (TCR) comprising an α chain comprising one or more of the following amino acid residues:

glutamine or glycine at position 1, isoleucine or lysine at position 2, valine at position 4, aspartic acid, proline, glutamine or glutamic acid at position 10, serine at position 12, leucine or valine at position 13, alanine or proline at position 14, asparagine at position 17, tyrosine at position 25, histidine at position 43, glutamine at position 44, tyrosine, asparagine or proline at position 45, leucine or serine at position 46, methionine or phenylalanine at position 54, alanine or serine at position 77, alanine at position 78, threonine or valine at position 80, lysine at position 82, serine, alanine or threonine at position 84, arginine or threonine at position 85, valine or phenylalanine at position 87, histidine or leucine at position 88, leucine or glutamine at position 95, glutamic acid or threonine at position 97, and serine at position 99; wherein the one or more amino acid residues is not present in the corresponding germline TCR amino acid sequence.

Paragraph 5. A polynucleotide encoding a TCR α chain according to paragraph 4.

Paragraph 6. A polynucleotide according to paragraph 6 which further comprises a polynucleotide encoding an β chain separated from the polynucleotide encoding the α chain by an internal self-cleaving sequence or an internal ribosome entry site.

Paragraph 7. A vector comprising a polynucleotide according to paragraph 5 or 6.

Paragraph 8. A cell comprising a polynucleotide according to paragraph 5 or 6 or a vector according paragraph 7.

Paragraph 9. A pharmaceutical composition comprising a TCR according to paragraph 4, a polynucleotide according to paragraph 6, a vector according to paragraph 7 or a cell according to paragraph 8.

Paragraph 10. A TCR, polynucleotide, vector, cell or pharmaceutical composition according to paragraphs 4 to 9 for use in treating and/or preventing a disease.

Paragraph 11. A vector according to paragraph 7 or cell according to paragraph 8 for use in the manufacture of a medicament for treating and/or preventing a disease.

Paragraph 12. A method for treating a disease which comprises the step of administering a TCR, polynucleotide, vector, cell or pharmaceutical composition according to any of paragraphs 4 to 9 to a subject.

Paragraph 13. A method for producing cell according to paragraph 8 which comprises the step of transducing a cell in vitro or ex vivo with a vector according to paragraph 7.

Paragraph 14. Use of an engineered TCR according to paragraph 5; a polynucleotide according to paragraph 6 or a vector according to paragraph 7 to increase the cell surface expression of the TCR.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Determination of Residues Associated with Dominant TCR Expression

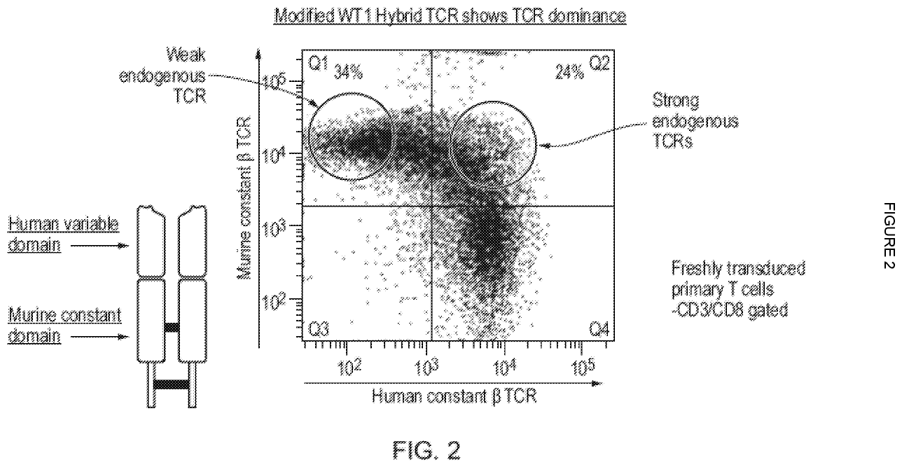

Peripheral blood T cells from two healthy donors were transduced with retroviral vectors encoding strongly expressed synthetic TCRs containing human variable domains and murine constant domains with an artificial disulphide bond between residue 48 of the α chain and residue 79 of the β chain. Antibodies specific for the human constant β domain were used to assess expression of endogenous human TCRs, and antibodies specific for the murine constant β domain were used to assess expression of the introduced synthetic TCRs. Flow cytometric sorting was used to purify transduced donor T cells that a dominant endogenous TCR that was co-expressed with the introduced TCR, or expressed a weak endogenous TCR that was undetectable in T cells that expressed the introduced TCR (see FIG. 2).

Next generation sequencing was employed to generate TCR libraries containing more than 130,000 distinct endogenous TCR clonotypes with a dominant or a weak expression phenotype (protocol for library generation as described by Uddin et al.; Methods Mol Biol 1884, 15-42 (2019)). Statistical analysis of these libraries revealed enrichment of a number of amino acid residues at various positions in the framework regions of the TCR variable domains, in particular in the Vβ domain.

The Vβ analysis showed over-representation of certain amino acids at 63 of the 77 framework positions, and the Vα analysis revealed over-representation at 68 of 77 framework positions in TCRs with a dominant expression profile.

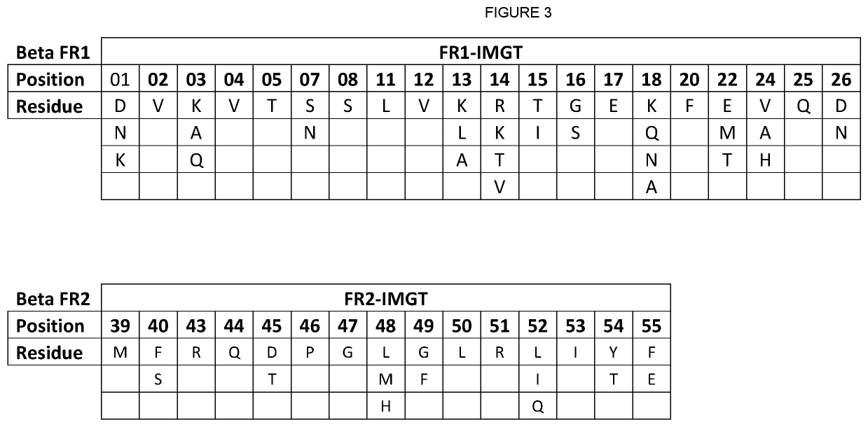

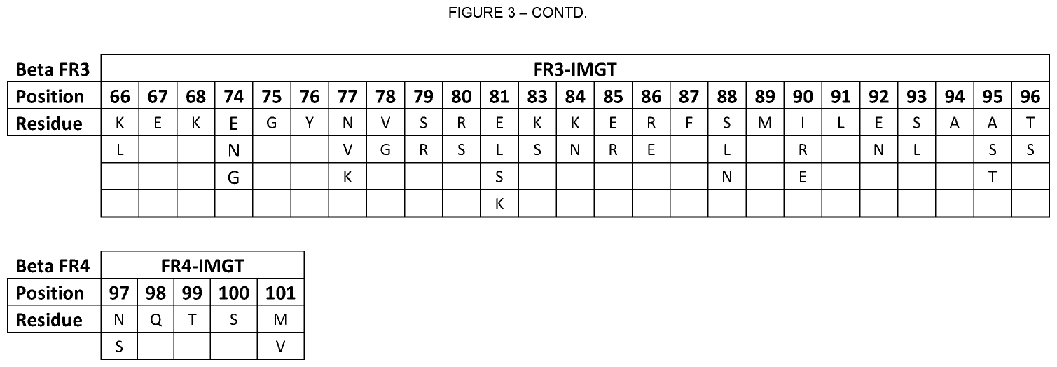

Residues that are highly significantly over-represented in the Vβ domain of dominant TCRs are shown in FIG. 3.

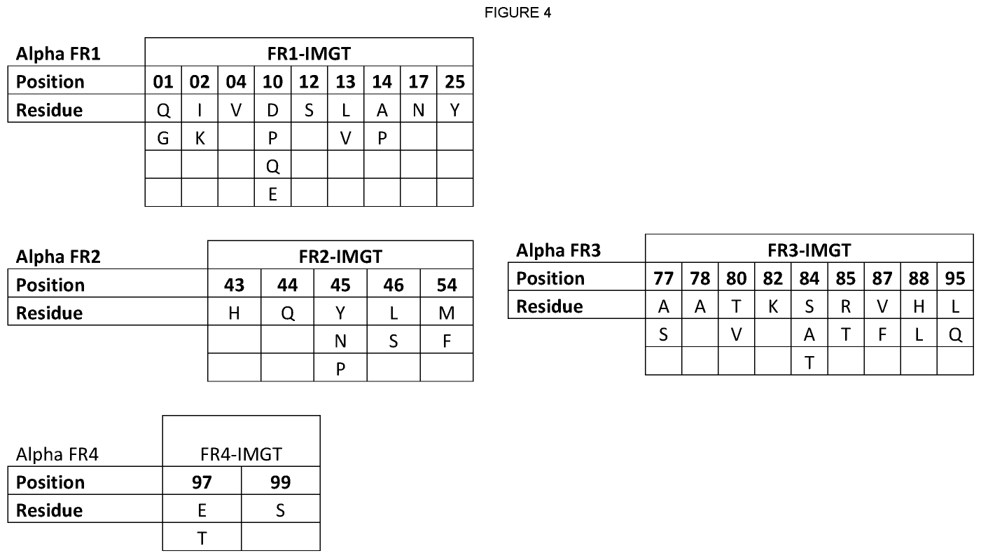

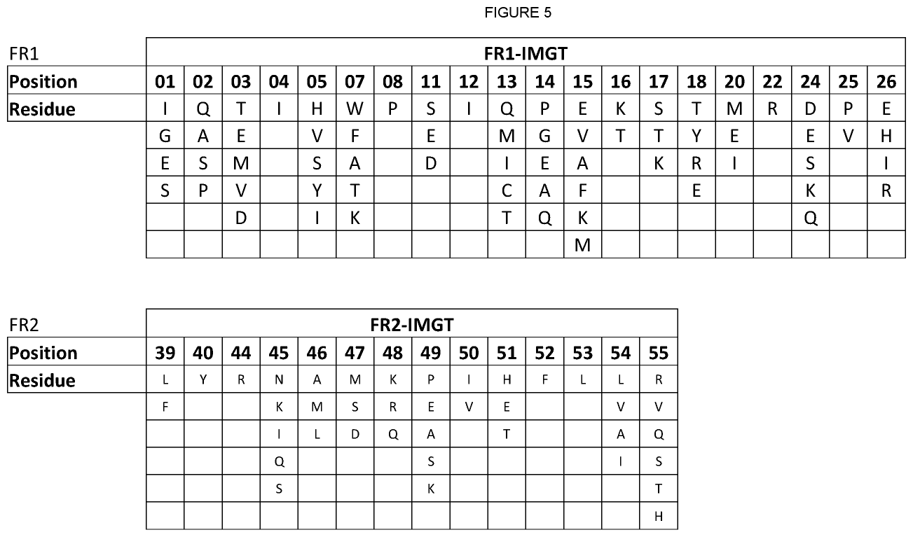

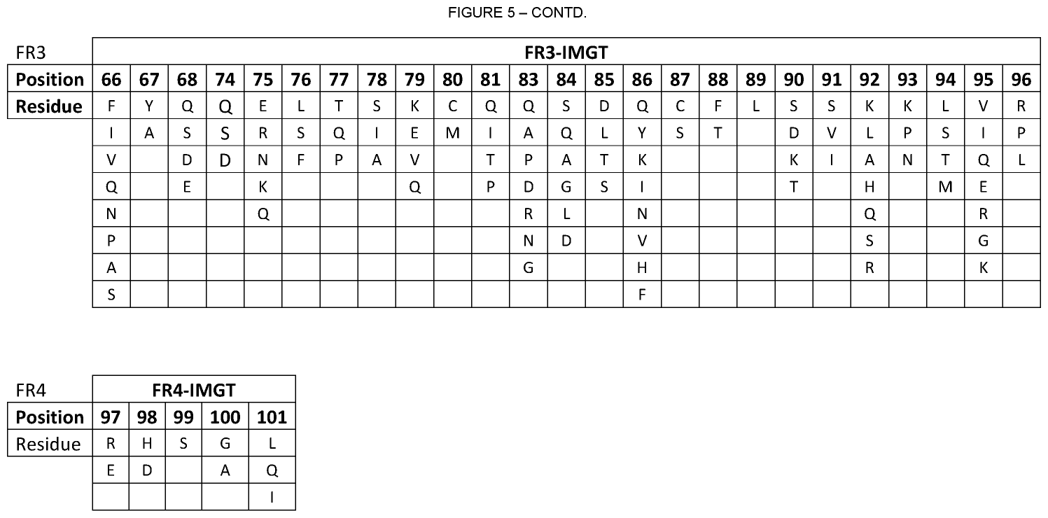

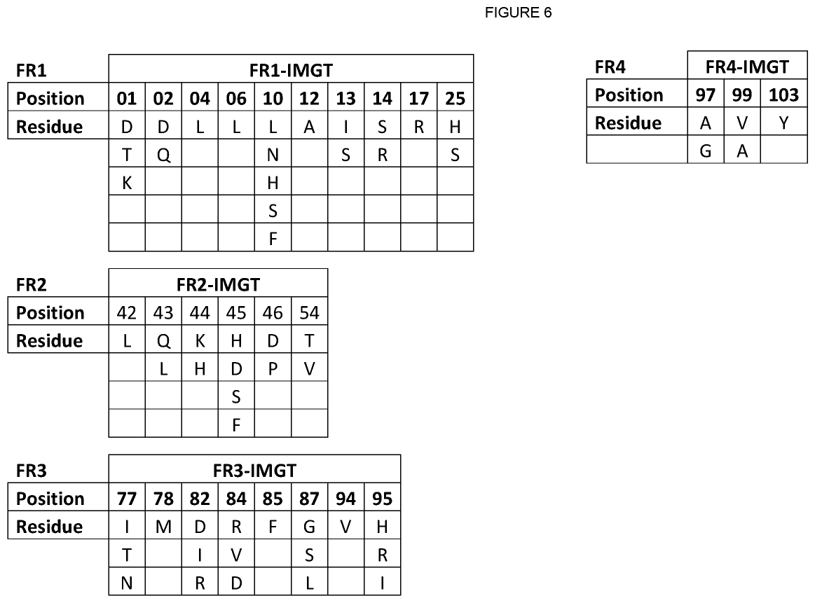

Residues that are highly significantly over-represented in the Vα domain of dominant TCRs are shown in FIG. 4.

### Materials and Methods

TCR library generated by next generation sequencing: at each position of the framework regions we compared the frequency of all amino acids in the dominant and in the weak TCR libraries using either Fisher's exact test performed on each donor independently, or the Cochran-Mantel-Haenszel test (CMH) performed on both donors together. The results were adjusted for multiple comparisons using the Bonferroni correction.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

1. A method for increasing the cell surface expression and/or reducing mispairing of a TCR which comprises the steps of:
(i) providing a TCR β chain sequence;
(ii) determining an amino acid residue of the TCR β chain at one or more positions selected from one or more of:
A) position 66; 67; 68; 74; 75; 76; 77; 78; 79; 80; 81; 83; 84; 85; 86; 87; 88; 89; 90; 91; 92; 93; 94; 95; 96;
B) position 1; 2; 3; 4; 5; 7; 8; 11; 12; 13; 14; 15; 16; 17; 18; 20; 22; 24; 25; 26;
C) position 39; 40; 44; 45; 46; 47; 48; 49; 50; 51; 52; 53; 54; 55;
D) position 97; 98; 99; 100; or 101 of the β chain and
(iii) altering the amino acid residue at one or more of the positions listed in step (ii) to an amino acid residue selected from:
A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;
B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;
C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;
D) arginine or serine at position 97; glutamine at position 98; threonine at position 99; serine at position 100; and methionine or valine at position 101 of the β chain.

2. A method for selecting a TCR which comprises the steps of:
(i) providing one or more TCR β chain sequences; and
(ii) selecting a TCR amino acid sequence comprising one or more amino acid residues selected from one or more of:
A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;
B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;
C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;
D) arginine or serine at position 97; glutamine at position 98; threonine at position 99; serine at position 100; and methionine or valine at position 101 of the β chain.

3. A method for determining the strength of a TCR which comprises the steps of:
(i) providing a TCR β chain sequence; and
(ii) determining if the TCR sequence comprises one or more amino acid residues selected from one or more of:
A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;
B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;
C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;
D) arginine or serine at position 97; glutamine at position 98; threonine at position 99; serine at position 100; and methionine or valine at position 101 of the β chain.

4. A method according to any of claims 1 to 4 wherein the one or more altered amino acid residues are selected from:
lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain.

5. An engineered T cell receptor (TCR) comprising a β chain comprising one or more of the following amino acid residues:
A) lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain;
B) aspartic acid, asparagine or lysine at position 1; valine at position 2; lysine, alanine or glutamine at position 3; valine at position 4; threonine at position 5; serine or asparagine at position 7; serine at position 8; leucine at position 11; valine at position 12; lysine, leucine or alanine at position 13; arginine, lysine, threonine or valine at position 14; threonine or isoleucine at position 15; glycine or serine at position 16; glutamic acid at position 17; lysine, glutamine, asparagine or alanine at position 18; phenylalanine at position 20; glutamic acid, methionine or threonine at position 22; valine, alanine or histidine at position 24; glutamine at position 25; and aspartic acid or asparagine at position 26 of the β chain;
C) methionine at position 39; phenylalanine or serine at position 40; glutamine at position 44; aspartic acid or threonine at position 45; proline at position 46; glycine at position 47; leucine, methionine or histidine at position 48; glycine or phenylalanine at position 49; leucine at position 50; arginine at position 51; leucine, isoleucine or glutamine at position 52; isoleucine at position 53; tyrosine or threonine at position 54; and phenylalanine or glutamic acid at position 55 of the β chain;
D) arginine or serine at position 97; glutamine at position 98; threonine at position 99; serine at position 100; and methionine or valine at position 101 of the β chain;
wherein the one or more amino acid residues is not present in the corresponding germline TCR amino acid sequence.

6. An engineered T cell receptor (TCR) according to claim 5 wherein the one or more amino acid residues are selected from:
lysine or leucine at position 66; glutamic acid at position 67; lysine at position 68; glutamic acid, asparagine or glycine at position 74; glycine at position 75; tyrosine at position 76; asparagine, valine or lysine at position 77; valine or glycine at position 78; serine or arginine at position 79; arginine or serine at position 80; glutamic acid, leucine, serine or lysine at position 81; lysine or serine at position 83; lysine or asparagine at position 84; glutamic acid or arginine at position 85; arginine or glutamic acid at position 86; phenylalanine at position 87; serine, leucine or asparagine at position 88; methionine at position 89; isoleucine, arginine or glutamic acid at position 90; leucine at position 91; glutamic acid or asparagine at position 92; serine or leucine at position 93; alanine at position 94; alanine, serine or threonine at position 95; and threonine or serine at position 96 of the β chain.

7. A polynucleotide encoding a TCR β chain as according to claim 5 or 6.

8. A polynucleotide according to claim 7 which further comprises a polynucleotide encoding a TCR α chain separated from the polynucleotide encoding the β chain by an internal self-cleaving sequence or an internal ribosome entry site.

9. A vector comprising a polynucleotide according to claim 7 or 8.

10. A vector according to claim 9 which is a retrovirus vector, lentivirus vector or a transposon.

11. A cell comprising a polynucleotide according to claim 7 or 8 or a vector according claim 9 or 10.

12. A cell according to claim 11 which is a T-cell, a natural killer cell or a stem cell.

13. A pharmaceutical composition comprising a TCR according to claim 5 or 6, a polynucleotide according to claim 7 or 8, a vector according to claim 9 or 10 or a cell according to claim 11 or 12.

14. A TCR, polynucleotide, vector, cell or pharmaceutical composition according to claims 5 to 13 for use in treating and/or preventing a disease.

15. A vector according to claim 9 or 10 or cell according to claim 11 or 12 for use in the manufacture of a medicament for treating and/or preventing a disease.

16. A method for treating a disease which comprises the step of administering a TCR, polynucleotide, vector, cell or pharmaceutical composition according to any of claims 5 to 13 to a subject.

17. A method for producing cell according to claim 11 or 12 which comprises the step of transducing a cell in vitro or ex vivo with a vector according to claim 9 or 10.

18. Use of an engineered TCR according to any of claim 5 or 6; a polynucleotide according to claim 7 or 8 or a vector according to claim 9 or 10 to increase the cell surface expression of the TCR.
